(19) United States
(12) Patent Application Publication
(10) Pub. No.: US 2009/0247532 A1
(43) Pub. Date: Oct. 1, 2009

(54) Crystalline polymorph of sitagliptin phosphate and its preparation

(75) Inventors: Cai Gu Huang, Guangzhou (CN); Hui Min He Huang, Guangzhou (CN)

Correspondence Address: Mae De Ltd., P. O. Box 193, Nothborough, MA, 01532, US

(73) Assignee: Mae De Ltd.

(21) Appl. No.: 12/321,146

(22) Filed: Jan. 21, 2009

Related U.S. Application Data
(60) Provisional application No. 61/072,107, filed on Mar. 28, 2008.

(51) Int. Cl. A61K 31/4985; C07D 487/04

(52) U.S. Cl. 514/249; 544/350

(57) ABSTRACT

The present invention is directed to a novel polymorph form of crystalline sitagliptin phosphate, named as Form V herein. The present invention further provides processes for preparations of Form V, pharmaceutical composition comprising Form V and its use in therapy. Form V can be prepared from recrystallizing sitagliptin phosphate in a mixture of methanol and water, a mixture of acetone and water, or from distillation of a mixture of organic solvents and water followed by recrystallization in the remaining aqueous solution.

This application claims the benefit of priority to U.S. Provisional Patent Application Ser. No. 61/072,107 filed on Mar. 28, 2008, the disclosure of which is hereby incorporated by reference.

### FIELD OF THE INVENTION

The present invention relates to a novel crystalline polymorphic form of sitagliptin phosphate, to processes for its preparations, pharmaceutical composition comprising such material and its use in therapy.

### BACKGROUND OF THE INVENTION

Sitagliptin phosphate, its chemical name is dihydrogenphosphate salt of (2R)-4-oxo-4-[3-(trifluoromethyl)-5,6-dihydro[1,2,4]triazolo[4,3-α]pyrazin-7(8H)yl]-1-(2,4,5-trifluorophenyl)butan-2-amine, has the following structural formula:

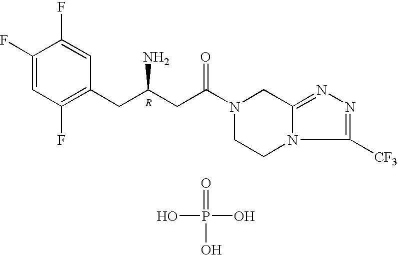

Sitagliptin is disclosed in WO 03/004498 and U.S. Pat. No. 6,699,871. Sitagliptin phosphate salt is disclosed in US patent application 2005/0032804. Sitagliptin or sitagliptin phosphate is a dipeptidyl peptidase-IV (DPP-IV) inhibitor and is useful for the treatment and prevention of Type 2 diabetes, hyperglycemia, insulin resistance, obesity, and high blood pressure.

U.S. Patent application 2005/0032804 discloses a (2R)-4-oxo-4-[3-(trifluoromethyl)-5,6-dihydro[1,2,4]triazolo[4,3-α]pyrazin-7(8H)yl]-1-(2,4,5-trifluorophenyl)butan-2-amine dihydrogenphosphate monohydrate, also known as sitagliptin phosphate monohydrate. The commercial JANUVIA tablets contain sitagliptin phosphate monohydrate.

U.S. Patent application 2006/0287528 discloses three polymorphic forms (Form I, Form II and Form III) of anhydrous sitagliptin phosphate and crystalline solvates of sitagliptin phosphate (e.g., ethanol solvate). This publication reports that Form II is a desolvated anhydrate form, which is metastable and converts to anhydrous forms such as Form I or Form III or mixtures thereof in about 2 hours at about 45° C. The ethanol solvate is also not a stable form and can be converted to desolvated Form II by (a) drying with nitrogen flow over the sample for about 5 hours at about 25° C. or (b) drying in vacuum for about 5 hours at about 25° C. Upon grinding or compaction of Form I, Form I can be converted into Form III. However, Form III is a stable form at low temperature and is stable below 34° C.

U.S. Patent application 2007/0021430 (or WO 2005/030127) discloses an anhydrate polymorph form of sitagliptin phosphate (Form IV). Form IV is prepared by heating sitagliptin phosphate monohydrate above 58° C. for about 8 hours. This publication reports that Form IV is also a metastable form and it converts into a crystalline sitagliptin phosphate monohydrate slowly under ambient conditions and rapidly under high relative humidity (98%) condition at room temperature.

U.S. Patent application 2007/028194 discloses a composition comprising an amorphous form of sitagliptin phosphate, which is obtained by freeze-drying sitagliptin phosphate in aqueous solutions. Amorphous material is usually less stable, both chemically and physically, relative to crystalline forms.

New crystalline polymorphic forms of a drug substance may display different melting point, hygroscopicity, stability, solubility and/or dissolution rate, crystallinity, crystal habits, bioavailability, toxicity and formulation handling characteristics, which are among the numerous properties that need to be considered in preparing medicament that can be effectively administered. Therefore, the regulatory agencies require a definitive control of polymorphic form of the active component in solid pharmaceutical dosage forms.

The known polymorphic forms of sitagliptin phosphate discovered so far are not stable (e.g., metastable) under ambient or storage conditions, contain high water content or tend to convert to other polymorphic forms under normal storage and manufacturing conditions. The drawbacks of known polymorphic forms of sitagliptin phosphate render them not suitable or less useful or favorable for preparing pharmaceutical formulations or bulk handling.

Accordingly, there is an ongoing need to search new polymorphic forms of sitagliptin phosphate that may have better stability and good material flow character, lower water contents, and may offer advantages for preparing reproducible pharmaceutical formulations. The novel and new polymorphic form of sitagliptin phosphate in the present invention help fulfill this and other needs.

### SUMMARY OF THE INVENTION

The inventors have now surprisingly discovered a novel crystalline polymorphic form (termed as Form V in the present invention) of sitagliptin phosphate, which are more thermodynamically stable, less static and better flow character and particularly suitable for bulk preparation, handling and formulation advantages. Therefore, the current invention is generally directed to a novel polymorphic form, namely Form V, of sitagliptin phosphate. Additionally, efficient, economical and reproducible processes are found for the preparation of Form V and Form I of sitagliptin phosphate.

Thus as a first aspect, the present invention provides a novel polymorphic form (Form V) of (2R)-4-oxo-4-[3-(trifluoromethyl)-5,6-dihydro[1,2,4]triazolo[4,3-α]pyrazin-7(8H)yl]-1-(2,4,5-trifluorophenyl)butan-2-amine dihydrogenphosphate (sitagliptin phosphate).

In another aspect, the present invention provides a composition comprising (a) polymorph Form V of sitagliptin phosphate and (b) a crystalline, hydrate, solvate, amorphous, polymorph Form I, Form II, Form III, Form IV or other polymorphic forms of sitagliptin phosphate other than Form V, wherein the total weight of sitagliptin phosphate in the composition is the sum of (a) and (b).

In another aspect, the present invention provides a process for preparing novel polymorph Form V of sitagliptin phosphate by recrystallizing sitagliptin phosphate in water, a mixture of organic solvents and water, a mixture of methanol and water or a mixture of acetone and water, followed by isolating and drying the product.

In a yet another aspect, the present invention provides processes for preparing Form I of sitagliptin phosphate by recrystallizing sitagliptin phosphate in a mixture of acetonitrile, dimethylacetamide (DMA) and water, or a mixture of n-butyl alcohol and water, or a mixture of isopropanol, acetone and water, followed by isolating and drying the product.

In another aspect, the present invention accordingly provides a pharmaceutical composition comprising Form V of sitagliptin phosphate and one or more pharmaceutically acceptable diluents or carriers and, optionally, one or more other physiologically active agents.

In a still aspect, the present invention provides a method for the use of Form V of sitagliptin phosphate for the treatment and/or prophylaxis of patients suffering from Type 2 diabetes, hyperglycemia, insulin resistance, obesity, and high blood pressure, and certain complications thereof.

### BRIEF DESCRIPTION OF THE ACCOMPANYING DRAWINGS

FIG. 1: X-ray powder diffraction (X-RPD) pattern of Form V of sitagliptin phosphate prepared in Example 1.

FIG. 2: X-ray powder diffraction (X-RPD) pattern of Form V of sitagliptin phosphate prepared in Example 2.

FIG. 3: X-ray powder diffraction (X-RPD) pattern of Form V of sitagliptin phosphate prepared in Example 2.

FIG. 4: Differential scanning calorimetry (DSC) of Form V of sitagliptin phosphate prepared in Example 1.

FIG. 5: Differential scanning calorimetry (DSC) of Form V of sitagliptin phosphate prepared in Example 2.

FIG. 6: Thermogravimetric analysis (TGA) of Form V of sitagliptin phosphate prepared in Example 2.

FIG. 7: Thermogravimetric analysis (TGA) of Form V of sitagliptin phosphate prepared in Example 1.

FIG. 8: FT-IR spectrum of Form V of sitagliptin phosphate prepared in Example 1 or Example 2.

FIG. 9: X-ray powder diffraction (X-RPD) pattern of Form I of sitagliptin phosphate prepared in Example 4.

FIG. 10: Differential scanning calorimetry (DSC) of Form I of sitagliptin phosphate prepared in Example 4.

### DETAILED DESCRIPTION OF THE INVENTION

Unless otherwise indicated, the following definitions are set forth to illustrate and define the meaning and scope of the various terms used to describe the invention herein.

The term “polymorphic form, polymorph, polymorph form or crystalline polymorph of sitagliptin phosphate” in the present invention refers to a crystal modification of sitagliptin phosphate, which can be characterized by analytical methods such as X-ray powder diffraction pattern, thermogravimetric analysis (TGA), differential scanning calorimetry (DSC), by its melting point or other techniques.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” or “pharmaceutical formulation” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The term “composition” is intended to encompass a particular pure polymorphic (or phase pure) form or a mixture of a particular polymorphic form along with other polymorph forms, solvate, amorphous form, hydrate or co-crystals. The composition may comprise a particular polymorphic form from a trace amount or less than 0.1% to 100% (weight by weight) based on the total amount of sitagliptin phosphate in the composition.

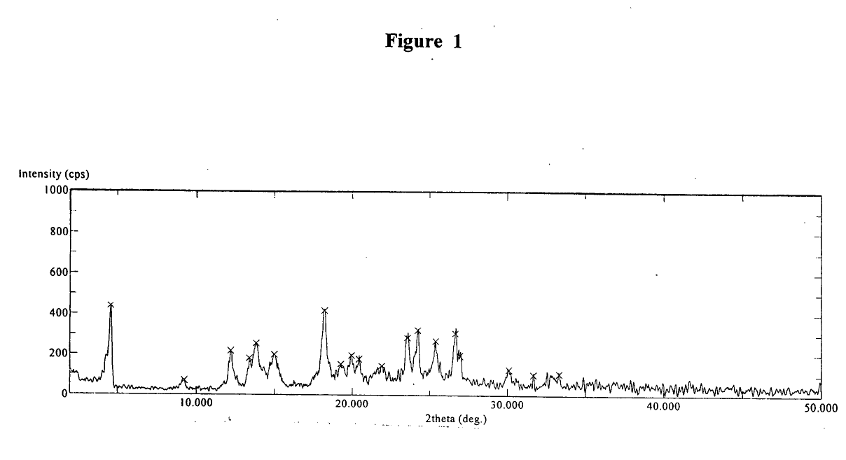

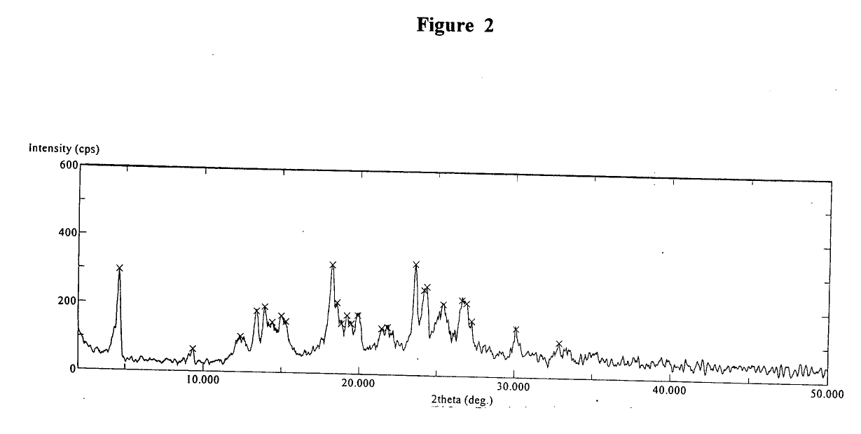

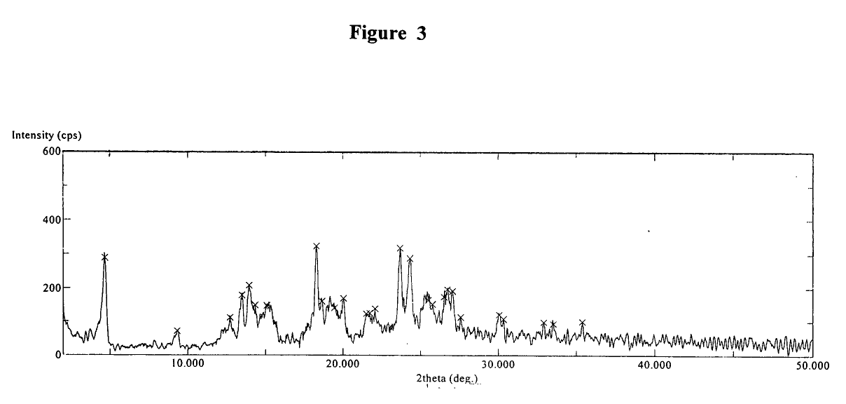

According to one aspect of the present invention, there is provided a novel polymorphic form of (2R)-4-oxo-4-[3-(trifluoromethyl)-5,6-dihydro [1,2,4]triazolo[4,3-a]pyrazin-7(8H)yl]-1-(2,4,5-trifluorophenyl)butan-2-amine dihydrogenphosphate (sitagliptin phosphate), designated as Form V herein, having an X-ray powder diffraction pattern (X-RPD), or substantially the same X-ray powder diffraction pattern, as shown in FIG. 1, FIG. 2 or FIG. 3. More particularly, polymorphic Form V of sitagliptin phosphate according to the present invention can be characterized as having an X-ray diffraction pattern with characteristic peaks (expressed in 2θ±0.2° 2θ) at one or more of the following positions: 4.60, 9.32, 12.38, 13.40, 13.92, 18.24, 23.60, 24.36, 25.40 or 26.60. Form V of sitagliptin phosphate according to the present invention can be further characterized as having an X-ray diffraction pattern with characteristic peaks (expressed in 2θ±0.2° 2θ) at one or more of the following positions: 4.60, 9.32, 12.38, 13.40, 13.92, 14.40, 14.98, 15.28, 18.24, 18.56, 18.84, 19.20, 19.48, 19.90, 21.44, 21.82, 23.60, 24.16, 24.36, 25.40, 26.60, 26.90, 27.24, 30.10 or 32.88.

Characterizing data for Form V of sitagliptin phosphate according to the present invention as obtained by X-ray powder diffraction is substantially the same as shown in FIG. 1, FIG. 2 or FIG. 3 and Table 1.

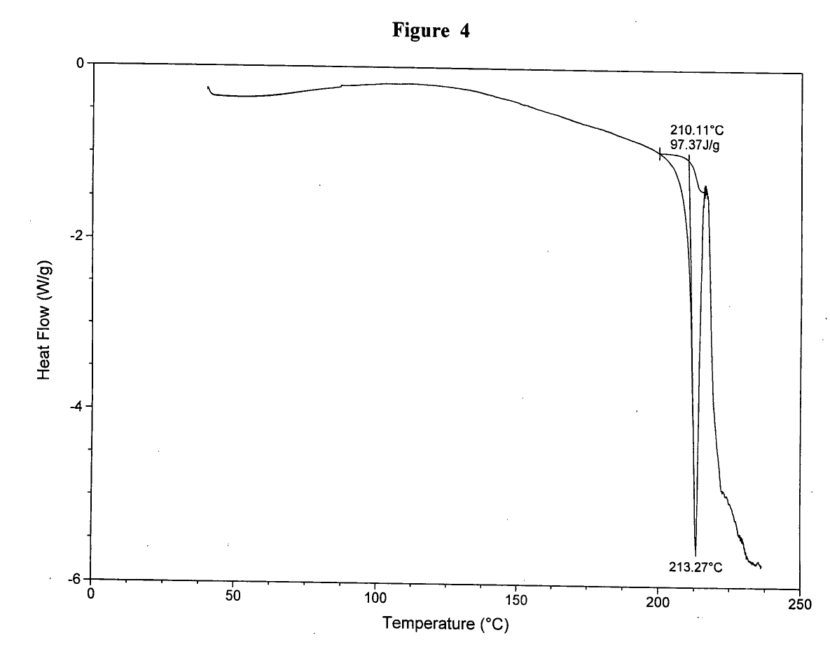

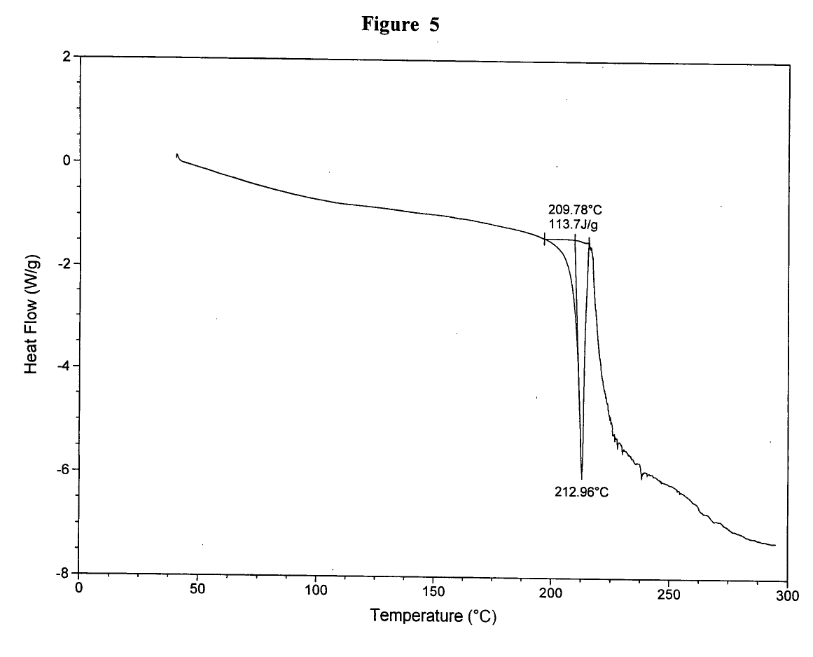

Further characterizing data for Form V of sitagliptin phosphate according to the present invention as obtained by differential scanning calorimetry (DSC) is substantially the same as shown in FIG. 4 or FIG. 5, and it provides an endothermic peak at around 210-214° C. (typically about 212° C.).

TABLE 1
Characteristic X-ray Powder Diffraction Pattern Peaks
(expressed in 2θ ± 0.2° 2θ) and Relative Intensities of
Diffraction Lines for Form V of Sitagliptin Phosphate

| Degree 2θ ± 0.2° 2θ | I/Iₒ |
|---|---|
| 4.60 | 92 |
| 9.32 | 20 |
| 12.38 | 33 |
| 13.40 | 56 |
| 13.92 | 60 |
| 14.40 | 46 |
| 14.98 | 52 |
| 15.28 | 47 |
| 18.24 | 98 |
| 18.56 | 65 |
| 18.84 | 46 |
| 19.20 | 53 |
| 19.48 | 46 |
| 19.90 | 54 |
| 21.44 | 42 |
| 21.82 | 43 |
| 23.60 | 100 |
| 24.16 | 78 |
| 24.34 | 81 |
| 25.40 | 65 |
| 26.60 | 69 |
| 26.90 | 66 |
| 27.24 | 50 |
| 30.10 | 44 |
| 32.88 | 32 |

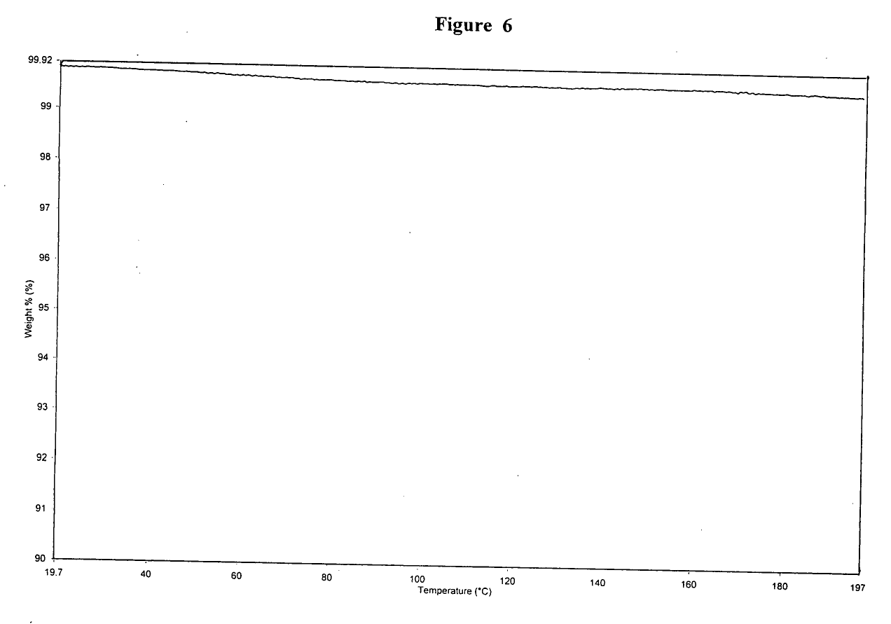

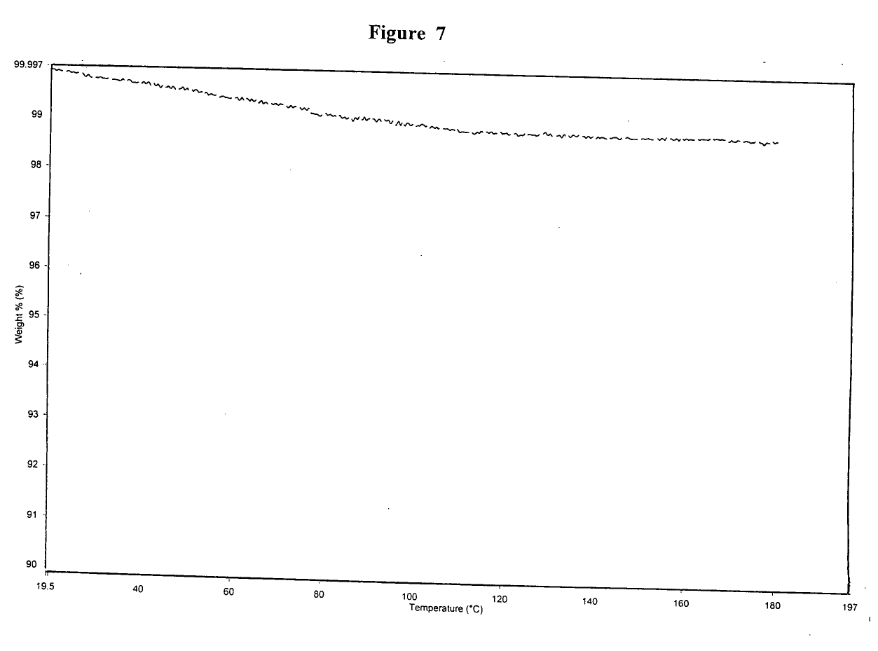

Still further characterizing data for polymorphic Form V of sitagliptin phosphate according to the present invention obtained by thermogravimetric analysis (TGA) is substantially the same as shown in FIG. 6 or FIG. 7, and it provides a loss of water at less than or about 0.1% to 0.5% w/w from 80° C. to 160° C. When the vacuum drying time is about 7 hours, the water content in the product is about 0.5% w/w as shown in Example 1. When the vacuum drying time is 14 hours or longer, the water content in the product is only about 0.1% w/w or less, as shown in Example 2 or Example 3. Without binding any theory, the applicant thinks that the small amount of water residue present in the product may not be a part of crystalline sitagliptin phosphate structure, and it may be free water, which can be removed by vacuum drying. The sitagliptin phosphate monohydrate would theoretically provide a loss of water at about 3.4% w/w and a semi-hydrate of sitagliptin phosphate would theoretically provide a loss of water at about 1.7% w/w. Therefore, Form V according to the present invention is a crystalline sitagliptin phosphate anhydrate.

The crystalline anhydrate sitagliptin phosphate form (Form V) according to the present invention is thermally more stable than other known forms of sitagliptin phosphate. For instance, Form V does not undergo a phase transformation even heating up to 41° C. Additionally, Form V has lower water content, good material flow characteristic and better chemical stability. These favorable characters render Form V a superior polymorphic form for pharmaceutical formulation and bulk handling of sitagliptin phosphate.

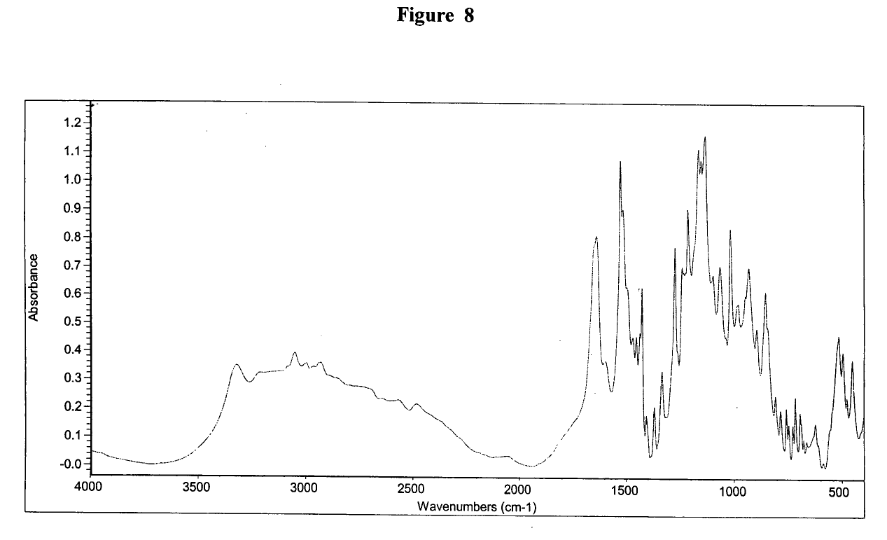

Further characterizing data for polymorphic Form V of sitagliptin phosphate according to the present invention obtained by the Fourier transform infrared (FT-IR) spectrum is substantially the same as shown in FIG. 8, and it contains peaks at one or more of the following positions of about 3322, 3050, 1638, 1526, 1454, 1427, 1371, 1335, 1275, 1214, 1165, 1160, 1135, 1066, 1017, 982, 932, 895, 854, 808, 716, 516, 496 or 453 cm⁻¹.

In one favored aspect, the polymorph Form V of sitagliptin phosphate provides X-ray powder diffraction (X-RPD) pattern substantially in accordance with FIG. 1, FIG. 2 or FIG. 3 and Table 1.

In one favored aspect, the Form V of sitagliptin phosphate provides differential scanning calorimetry (DSC) substantially in accordance with FIG. 4 or FIG. 5.

In one still favored aspect, the Form V of sitagliptin phosphate provides thermogravimetric analysis (TGA) substantially in accordance with FIG. 6 or FIG. 7.

In one favored aspect, the Form V of sitagliptin phosphate provides the Fourier transform infrared (FT-IR) substantially in accordance with FIG. 8.

The present invention encompasses Form V of sitagliptin phosphate isolated in pure form or in a mixture as a solid composition when admixed with other materials, for example the other known polymorphic forms (i.e. amorphous form, solvates, Form I, Form II, Form III, Form IV, or other forms) of sitagliptin phosphate or any other materials.

Thus in one aspect there is provided Form V of crystalline sitagliptin phosphate in isolated solid form.

In a further aspect there is provided Form V of sitagliptin phosphate in phase pure form. The phase pure form means that Form V is over 95% (w/w), preferably over 98% (w/w), more preferably over 99% (w/w %) and most preferably over 99.5% (w/w) or over 99.9% (w/w).

More specifically, the present invention provides that Form V of sitagliptin phosphate is in the form of a composition or a mixture of Form V along with one or more other crystalline, solvate, amorphous, or other polymorphic forms or their combinations thereof of sitagliptin phosphate. Such a composition may be a drug substance or an active ingredient in pharmaceutical compositions or formulations. For example, such composition may comprise polymorphic Form V along with one or more other polymorphic forms of sitagliptin phosphate, such as amorphous form, hydrate, solvates, polymorph Form I, Form II, Form III, Form IV and/or other forms or their combinations thereof. More specifically, the composition may comprise from trace amounts up to 100% Form V, or any amount in between—for example, the composition may comprise less than 0.1%, 0.5%, 1%, 2%, 5%, 10%, 20%, 30%, 40% or 50% by weight of Form V based on the total amount of sitagliptin phosphate in the composition. Alternatively, the composition may comprise at least 50%, 60%, 70%, 80%, 90%, 95%, 97%, 98%, 99%, 99.5% or 99.9% by weight of Form V based on the total amount of sitagliptin phosphate in the composition.

In yet a further aspect there is provided Form V of sitagliptin phosphate in crystalline form.

In a preferred aspect, the particle size of polymorphic Form V of sitagliptin phosphate in the present invention has the median value of the volume mean diameter of the particles within the range of 0.01 μm-450 μm, preferably 5-250 μm, and most preferably 50-150 μm. Such particles are better in chemical and physical stability, good material flow characteristics, improving the uniformity of dosage forms and thus suitable for bulk preparation and formulation advantages.

According to another aspect, the present invention provides a process for preparing polymorph Form V of sitagliptin phosphate. Polymorph Form V may be prepared by crystallization from a crystallization solvent containing sitagliptin phosphate. As used herein, the term “crystallization solvent” means a solvent or combination of solvents from which sitagliptin phosphate is preferentially crystallized as polymorph Form V. Representative crystallization solvents for preparation of Form V include water, tetrahydrofuran (THF), methanol, acetone, acetonitrile, isopropanol, n-butanol, dichloromethane and combinations thereof. In a preferred aspect, the crystallization solvent comprises water, to which methanol, acetone, isopropanol, acetonitrile, n-butanol or combinations thereof is gradually added.

In a preferred aspect, Form V of sitagliptin phosphate may be prepared by slurring starting material, crude or pure sitagliptin phosphate, anhydrate or solvate, which can be obtained according to the procedures described in U.S. patent application 2005/0032804 with a polar organic solvent or a mixture of two or more polar organic solvents under heat. The sitagliptin phosphate is soluble in water, but not soluble in polar organic solvents. The preferred polar organic solvent is methanol or acetone. The concentration of sitagliptin phosphate within the solution may range from about 0.1% by weight to the saturation point. This concentration will, of course, vary depending upon the temperature at which the co-solvent solution is held, with warmer temperatures generally allowing for the preparation of more concentrated solutions of sitagliptin phosphate. Preferably, the concentration (w/w %) of sitagliptin phosphate starting material in solution is about 0.5-15%, preferably about 1-10%, more preferably about 1.5-5%. The volume ratio of water to methanol or acetone is about 1:0.1-200, preferably about 1:2-30, more preferably about 1:3-10, most preferably about 1:4-6. Water is then added into the above suspension, and the mixture is heated, suitably to a temperature in the range of from about 45° C. to 85° C., such as about 50° C. to 75° C., for example about 70° C. until all solid materials are dissolved. The clear and hot solution is allowed to cool down to ambient temperature, and the cooled solution is kept at about −25° C.-20° C. for crystallization, preferably at about −20° C.-0° C., and more preferably at about −18° C. The crystal Form V of sitagliptin phosphate is formed over a period of two to ten days, and the crystal Form V is recovered from the solvent by filtration. The obtained crystal From V can be dried under a vacuum oven at about 20° C.-60° C., preferably at about 35° C.-50° C., more preferably at about 35° C.-45° C., and most preferably at 38-42° C. for about 10-40 hours to remove the solvent residues.

In a preferred aspect, Form V of sitagliptin phosphate may be prepared by slurring starting material, crude or pure sitagliptin phosphate, anhydrate or solvate, which can be obtained according to the procedures described in U.S. patent application 2005/0032804 with a mixture of water and one or more organic solvents selected from a group consisting of n-butanone, THF, ethyl acetate, acetone, dichloromethane, acetonitrile or methanol under heat. The volume ratio of water to a mixture of one or more organic solvents selected from a group consisting of n-butanone, THF, ethyl acetate, acetone, dichloromethane, acetonitrile or methanol is about 1:0.1-200, preferably about 1:2-30, more preferably about 1:3-10, most preferably about 1:4-6. The mixture is heated, suitably to a temperature in the range of from about 45° C. to 85° C., such as about 50° C. to 75° C., for example about 70° C. until all solid materials are dissolved. The organic solvents in solution were removed by distillation under the reduced pressure at about 15-30 psi and at a temperature of about 45-60° C. The distillation is stopped when most of organic solvents are removed and a small amount of crystals are formed on the surface of the flask vessel. The hot solution is allowed to cool down to ambient temperature, and the cooled solution is kept at about −10° C.-60° C. for crystallization, preferably at about −0° C.-40° C., and more preferably at about 5° C. or 30° C. or ambient temperature. The crystal Form V of sitagliptin phosphate is formed over a period of 20 min to 24 hours, and the crystal Form V is recovered from the solvent by filtration. The obtained crystal From V can be dried under a vacuum oven at about 20° C.-60° C., preferably at about 35° C.-50° C., more preferably at about 35° C.-45° C. and most preferably at 38-42° C. for about 10-40 hours to remove the solvent residues.

Once obtained, crystals of polymorph Form V may be used as the nucleating agent or “seed” crystals for subsequent crystallizations of polymorph Form V from the crystallization solvent. In one embodiment, the crystallization solvent is formed by dissolving sitagliptin phosphate in hot methanol or acetone or other suitable crystallization solvents. The crystallization solvent is then seeded with crystals of polymorph Form V, cooled and filtered, resulting in polymorph Form V. In another embodiment, a crystallization solvent is formed by slurring sitagliptin phosphate in methanol or acetone or other appropriate solvents. The crystallization solvent is then seeded with crystals of polymorph Form V and filtered, resulting in polymorph Form V. Such seeding with crystals of polymorph Form V may take place at any time during the slurring process. Alternatively, seeding with crystals of polymorph Form V may take place prior to, or simultaneously with, addition of sitagliptin phosphate to the crystallization solvent.

Form V of crystalline sitagliptin phosphate as obtained above is characterized by X-ray powder diffraction pattern, substantially the same as shown in FIG. 1, FIG. 2, or FIG. 3 and Table 1.

Form V of sitagliptin phosphate as obtained above is characterized by differential scanning calorimetry (DSC), substantially the same as shown in FIG. 4 or FIG. 5.

The crystals of sitagliptin phosphate obtained from recrystallization in solvents as described in above processes may have different crystal habits (e.g., shape), water contents, surface area, bulk or tap density, or particle size, but they clearly still belong to a new and novel polymorphic form (Form V) of sitagliptin phosphate, as it is characterized and confirmed by X-ray powder diffraction pattern and DSC thermogram, TGA and FT-IR. The X-ray powder diffraction pattern of Form V is clearly different from that of other known forms such as sitagliptin phosphate monohydrate, Form I, Form II, Form III, Form IV and other solvates.

According to a further aspect, the present invention further provides a pharmaceutical composition, which comprises a prophylactically and a therapeutically effective amount of polymorph Form V of sitagliptin phosphate, substantially as hereinbefore described, together with one or more pharmaceutically acceptable carriers, diluents or excipients, additives, fillers, lubricants, solvents, binders or stabilizers, optionally, one or more other active ingredients.

Pharmaceutical compositions as provided by the present invention can be prepared by known procedures using well-known and readily available ingredients. In preparation of compositions as provided by the present invention, polymorph Form V of crystalline sitagliptin phosphate, substantially as hereinbefore described, can be mixed with one or more carriers, excipients, diluents, additives, fillers, lubricants, solvents, binders or stabilizers, optionally, one or more other active ingredients.

Pharmaceutical compositions as provided by the present invention can be in the form of tablets, pills, powders, lozenges, sachets, cachets, elixirs, suspensions, emulsions, solutions, syrups, aerosol, ointments soft and hard gelatin capsules, suppositories, sterile injectable solutions and sterile packaged powders containing, for example, up to 70% by weight of polymorph Form V, substantially as hereinbefore described.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl- and propylhydroxybenzoates, talc, magnesium stearate and mineral oil. The compositions can additionally include lubricating agents, wetting agents, and emulsifying and suspending agents, preserving agents, sweetening agents or flavoring agents.

According to a still another embodiment, the pharmaceutical composition comprises an effective dosage amount of sitagliptin phosphate, wherein sitagliptin phosphate comprises at least a certain percentage of polymorph Form V (based on the total amount of sitagliptin phosphate present in the composition—that is, the total amount of sitagliptin phosphate being 100%). In other words, at least a certain percentage of sitagliptin phosphate present within the pharmaceutical composition exists as polymorph Form V, with the remainder of sitagliptin phosphate being in a different form, including (but not limited to) polymorph Form I, Form II, Form III, Form IV, or any other crystalline, solvate or amorphous form(s). More specifically, trace amounts up to 100% Form V, or any amount in between—for example, the active ingredient or drug substance of sitagliptin phosphate in the pharmaceutical composition may comprise less than 0.1%, 0.5%, 1%, 2%, 5%, 10%, 20%, 30%, 40% or 50% by weight of Form V based on the total amount of sitagliptin phosphate in the pharmaceutical composition. Alternatively, the pharmaceutical composition may comprise at least 50%, 60%, 70%, 80%, 90%, 95%, 97%, 98%, 99%, 99.5% or 99.9% by weight of Form V based on the total amount of sitagliptin phosphate in the pharmaceutical composition.

The pharmaceutical compositions of the invention may be formulated so as to provide quick, extended, sustained or delayed release of polymorph Form V of sitagliptin phosphate, substantially as hereinbefore described, after administration to the patient by employing procedures well known in the art. The pharmaceutical compositions of the invention may be preferably formulated so as to provide quick (or immediate), delayed, extended or sustained release tablets consisting of polymorph Form V of sitagliptin phosphate, substantially as hereinbefore described as active ingredient and plus any additional excipients suitable for preparation of quick, delayed, extended or sustained release tablets.

According to one preferred aspect, the pharmaceutical composition is a quick release formulation. For example, a quick release formulation may comprise lactose or dicalcium phosphate as main diluents, crystalline polymorph Form V of sitagliptin phosphate as active ingredient, microcrystalline cellulose as a binder or filler, a disintegratant and a lubricant. The dose units are preferably coated with a film coating.

According to one preferred aspect, the pharmaceutical composition is an extended release formulation. For example, an extended release formulation may comprise spheroids comprised of crystalline polymorph Form V of sitagliptin phosphate, microcrystalline cellulose, and, optionally, hydroxypropylmethylcellulose. The spheroids are preferably coated with a film coating composition comprised of ethyl cellulose and hydroxypropylmethylcellulose.

According to another preferred embodiment, the pharmaceutical composition is a sustained release formulation (e.g., in the form of a tablet). The sustained release formulation may comprise crystalline polymorph Form V of sitagliptin phosphate, a release rate controlling excipient, and optionally other adjuvants. Suitable rate controlling excipients include, but not limited to, hydroxyalkyl cellulose, such as hydroxypropyl cellulose and hydroxypropyl methyl cellulose (HPMC); poly(ethylene) oxide; alkyl cellulose, such as ethyl cellulose and methyl cellulose; carboxymethyl cellulose; hydrophilic cellulose derivatives; carboxyvinylpolymers (e.g., Carbopol 971P), polyvinylpyrrolidone (PVP) derivatives and polyethylene glycol derivatives.

The sustained release pharmaceutical composition comprises about 1-500 mg of polymorphs Form V of sitagliptin phosphate and about 15% w/w to about 70% w/w of a release rate controlling pharmaceutical excipients. A preferred sustained release pharmaceutical composition comprises from about 50-300 mg of crystalline polymorphs Form V of sitagliptin phosphate and about 10% w/w to about 66% w/w of hydroxyprpopyl methylcellulose, methyl cellulose or ethyl cellulose. Typically, the sustained release formulation provides sustained therapeutically effective plasma levels over at least about 6 or 24 hour period. The peak serum levels during the 6 or 24 hour period are generally up to 5 to 200 ng/mL.

The pharmaceutical compositions are preferably formulated in a unit dosage form, each dosage containing from about 2 to about 500 mg, more usually about 20 to about 300 mg, of polymorph Form V of sitagliptin phosphate, substantially as hereinbefore described. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier.

A further aspect of the present invention relates to a method of treating or preventing patients suffering from Type 2 diabetes, hyperglycemia, insulin resistance, obesity, and high blood pressure, and certain complications thereof, comprising administering to a patient in need of such treatment an effective amount of a pharmaceutical composition comprising polymorph Form V of sitagliptin phosphate and a pharmaceutically acceptable carrier.

The present invention further provides polymorph Form V of sitagliptin phosphate, for use in the manufacture of a medicament for the treatment and/or prophylaxis of patients suffering from Type 2 diabetes, hyperglycemia, insulin resistance, obesity, and high blood pressure, and certain complications thereof.

The particular dose of polymorph Form V of sitagliptin phosphate, substantially as hereinbefore described, administered according to this invention will of course be determined by the particular circumstances surrounding the case, the route of administration, the particular condition being treated, and similar considerations.

Polymorph Form V of sitagliptin phosphate, substantially as hereinbefore described, can be administered by a variety of routes including the oral, rectal, transdermal, subcutaneous, intravenous, intramuscular or intranasal routes. A typical daily dose will contain from about 0.01 mg/kg to about 100 mg/kg of polymorph Form V of the present invention. Preferred daily doses will be about 0.01 to about 10 mg/kg, ideally about 0.1 to about 5 mg/kg.

Having thus described the invention with reference to particular preferred embodiments, those in the art can appreciate modifications to the invention as described and illustrated that do not depart from the spirit and scope of the invention as disclosed in the specification. The following examples are set to illustrate the invention, and aid to understanding the invention, but not intended to, and should not be construed to limit its scope in any way.

### EXPERIMENTAL

Thermogravimetric analysis (TGA) measurements were performed in a Pyris I TGA of Perkin-Elmer (TGA7) under nitrogen purge. The sample was heated from 20° C. to 200° C. at a scan rate of 10° C./minute.

DSC measurements were performed in a TA instrument with a sealed pan at a scan rate of 10° C./minute from 40° C. to 260° C. under nitrogen purge.

X-ray powder diffraction (X-RPD) data were obtained by ARL X-Ray powder diffractometer model XTRA-030. Scanning range 3-50 deg. 2 theta, continuous scan, rate 3 deg./min. The accuracy of peak positions was defined as +/−0.2 degrees due to such experimental differences as instrumentation and sample preparation etc.

### EXAMPLES

### Example 1

### Preparation of Polymorph Form V of Sitagliptin Phosphate

Sitagliptin phosphate (4.0 g) was suspended in about 50 ml boiling methanol (HPLC grade). To the suspension was added about 10 ml water and the suspension was heated up until all solid materials are dissolved. The resulting clear solution was then cooled down to ambient temperature and kept at 5° C. for recrystallization for overnight and no crystals were formed. The recrystallization continued at about −18° C. for three days. The resulting crystals were isolated by filtration and dried in vacuum oven at about 41° C. for 7 hours to give a white crystalline solid (about 2.5 g). DSC, TGA and X-ray diffraction pattern techniques were used to characterize the obtained product. DSC experiment of the obtained product showed an endothermic peak at about 213.27° C., as shown in FIG. 4. Powder X-ray diffraction pattern of the obtained product is shown in FIG. 1. The TGA, as shown in FIG. 7, indicated that the obtained product contains about or less than 0.5% w/w water.

### Example 2

### Preparation of Polymorph Form V of Sitagliptin Phosphate

Sitagliptin phosphate monohydrate (2.5 g) was suspended in 5 ml boiling methanol (HPLC grade), 5 ml n-butanone, 4 ml THF, 5 ml acetonitrile and 10 ml dichloromethane (HPLC grade). To the suspension was added about 7 ml water. The suspension was heated up until all solid materials were dissolved and the mixture became a homogeneous solution. The resulting clear solution was distilled at about 55° C. under the reduced pressure (about 20 psi). The majority of organic solvents were removed by distillation and a small amount of white crystals were formed on the surface of the flask vessel. These crystals were placed in to the aqueous solution to induce the recrystallization of sitagliptin phosphate, and the suspension was kept at ambient temperature for cooling and further recrystallization. After about 20-30 minutes, lots of crystals were formed. The recrystallization was continued at ambient temperature for 2 hours. The resulting crystals were isolated by filtration and dried in vacuum oven at about 41° C. for 14 hours to give a white crystalline solid (about 1.7 g). DSC, TGA and X-ray diffraction pattern techniques were used to characterize the obtained product. DSC experiment of the obtained product showed an endothermic peak at about 212.96° C., as shown in FIG. 5. Powder X-ray diffraction pattern of the obtained product is shown in FIG. 3. The TGA as shown in FIG. 6 indicated that the obtained product contains about or less than 0.1% w/w water.

The mother liquids from above recrystallization experiment were kept at 5° C. for further recrystallization for 2 days. The resulting crystals were isolated by filtration and dried in vacuum oven at about 25° C. for 24 hours and than at about 41° C. for 24 hours to give a white crystalline solid (about 0.4 g). DSC, TGA and X-ray diffraction pattern techniques were used to characterize the obtained product. DSC experiment of the obtained product showed an endothermic peak at about 212.19° C. Powder X-ray diffraction pattern of the obtained product is shown in FIG. 2. The TGA indicated that the obtained product contains about or less than 0.1% w/w water.

### Example 3

### Preparation of Polymorph Form V of Sitagliptin Phosphate

Sitagliptin phosphate (4.0 g) was suspended in 50 ml boiling acetone (HPLC grade). To the suspension was added about 7 ml water. The suspension was heated up until all solid materials are dissolved, and the mixture became a clear and homogeneous solution. The resulting solution was cooled down to ambient temperature and kept at 5° C. for recrystallization for overnight and no crystals were formed. The recrystallization continued at −18° C. for three days. The resulting crystals were isolated by filtration and dried in vacuum oven at about 41° C. for 24 hours to give a white crystalline solid (about 2.0 g). DSC and X-ray diffraction pattern techniques were used to characterize the obtained product. DSC experiment of the obtained product showed an endothermic peak at about 211.97° C. Powder X-ray diffraction pattern of the obtained product is substantially the same as shown in FIG. 1 or FIG. 2.

### Example 4

### Preparation of Polymorph Form I of Sitagliptin Phosphate

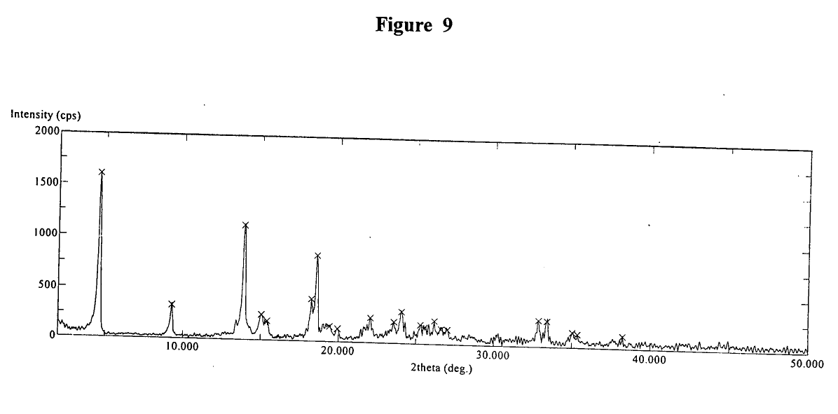

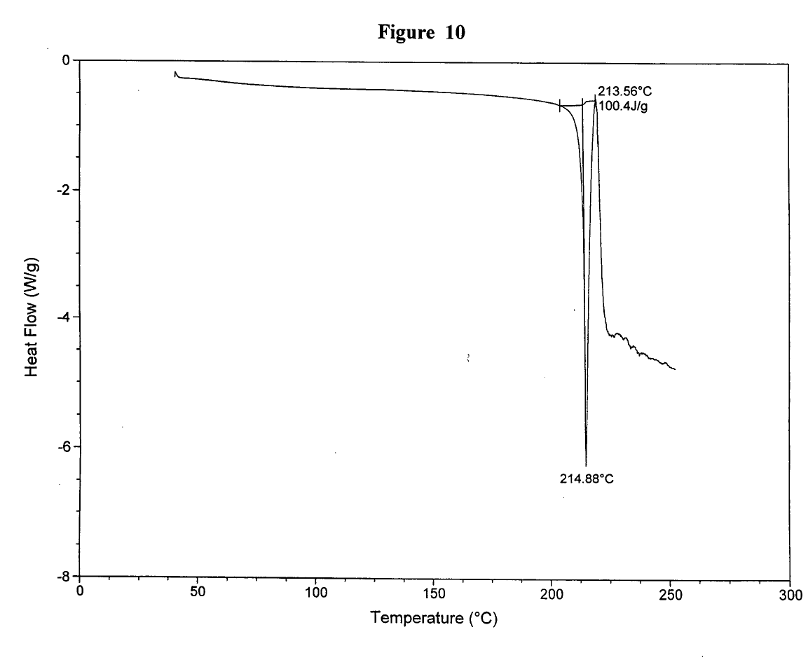

Sitagliptin phosphate (4.0 g) was suspended in 40 ml boiling acetonitrile (HPLC grade). To the suspension was added about 20 ml dimethyl acetamide and 20 ml water. The suspension was heated up until all solid materials are dissolved, and the mixture became a clear and homogeneous solution. The resulting clear solution was cooled down to ambient temperature and kept at 5° C. for recrystallization for three days. The resulting crystals were isolated by filtration and dried in vacuum oven at about 41° C. for 24 hours to give a white crystalline solid (about 2.6 g). DSC and X-ray diffraction pattern techniques were used to characterize the obtained product. DSC experiment of the obtained product showed an endothermic peak at about 214.88° C., as shown in FIG. 10. Powder X-ray diffraction pattern of the obtained product is shown in FIG. 9.

### Example 5

### Preparation of Polymorph Form I of Sitagliptin Phosphate

Sitagliptin phosphate (4.0 g) was suspended in 50 ml boiling n-butyl alcohol (HPLC grade). To the suspension was added about 8 ml water. The suspension was heated up until all solid materials are dissolved, and the mixture became a clear and homogeneous solution. The resulting solution was cooled down to ambient temperature and kept at 5° C. for recrystallization for overnight. The resulting crystals were isolated by filtration and washed with cold THF and ethanol, and then dried in vacuum oven at about 41° C. for 6 hours to give a white crystalline solid (about 2.9 g). DSC and X-ray diffraction pattern techniques were used to characterize the obtained product. DSC experiment of the obtained product showed an endothermic peak at about 214.39° C. Powder X-ray diffraction pattern of the obtained product is substantially the same as shown in FIG. 9.

### Example 6

### Formulation of Tablets Containing Crystalline Form V of Sitagliptin Phosphate

There were three major steps involved in manufacturing the tablets: (A) preparation of polymorphic form (Form V) of sitagliptin phosphate granular concentrate; (B) preparation of tablet core; (C) coating the tablet core. The amount of each ingredient included in the formulation is shown in Table 2 (quantity in gram).

### A: Preparation of Polymorph Form V of Sitagliptin Phosphate Granular Concentrate

The following ingredients (quantity in gram) were sifted through a clean screen (typically 0.066″): lactose anhydrous, dicalcium phosphate anhydrous, pregelatinized starch, sodium starch glycolate and microcrystalline cellulose.

The screened materials were transferred into a high shear (high-energy) mixer and blended for ten (10) minutes at 100 rpm. The blended material was granulated with purified water. The wet granules were passed through a screen (typically 0.132″), and dried in a fluid bed drier until loss on drying is less than 0.2-0.5% w/w.

TABLE 2
% Composition of Form V of Sitagliptin Phosphate (50%,
w/w) Granular Concentrate

| | Granular concentrate | | |
|---|---|---|---|
| | batch # | | |
| | 1 | 2 | 3 |
| Form V of sitagliptin | 100 | 100 | 100 |
| phosphate | | | |
| Lactose anhydrous | 50 | | 10 |
| Dicalcium phosphate | | 50 | 20 |
| anhydrous | | | |
| Hydroxypropyl methyl | | | 40 |
| cellulose (HPMC) | | | |
| Sodium starch glycolate | 10 | 10 | 10 |
| Pregelatinized starch | 10 | 20 | 10 |
| Microcrystalline cellulose | 30 | 20 | 10 |
| Purified water* | | | |

*Water was removed during the process

The dried granules were passed a screen (typically 0.039″) and blended using a tumble blender for 10 minutes at 12 rpm.

### B: Preparation of Tablet Core Comprising Form V of Sitagliptin Phosphate

The concentrated granules are placed into a tumble blender. About two thirds of the lactose or dicalcium phosphate is screened and added to the blender, and blended for ten (10) minutes. The microcrystalline cellulose, sodium starch glycollate, magnesium stearate and remaining lactose or dicalcium phosphate are screened and added to the blender. The mixtures are blended together for ten (10) minutes. The blended material was compressed on a Kikusui Libra tablet compression machine to a target weight of 300 mg for the 50 mg and 100 mg tablets.

### C: Preparation of Coated Tablet Comprising Form V of Sitagliptin Phosphate

The tablet cores are then transferred to a tablet-coating machine (pan coater). The tablet bed was pre-heated with warm air (approximately 60° C.). The pan speed

TABLE 3
% Composition of Tablet Core (quantity, mg per tablet)

| | Formulation batch# | | | | | |
|---|---|---|---|---|---|---|
| | 1 | | 2 | | 3 | |
| Dosage strength | 50 | mg | 100 | mg | 100 | mg |
| Form V of sitagliptin | 100 | | 200 | | 200 | |
| phosphate concentrate | | | | | | |
| granules | | | | | | |
| Lactose anhydrous | 150 | | 10 | | 20 | |
| Microcrystalline | 47 | | 87 | | 87 | |
| cellulose | | | | | | |
| Magnesium stearate | 3 | | 3 | | 3 | |
| Total weight | 296 | | 296 | | 296 | |
| Coating material | 4.0 | | 4.0 | | 4.0 | |
| Total weight of coated | 300.0 | | 300.0 | | 300.0 | |
| tablet | | | | | | |

was adjusted to 5-9 RPM before starting the spray cycle. The spray cycle was activated. The exhaust temperature was maintained between 40° C. and 50° C. throughout the cycle. After the proper amount of solution was applied, the coated tablets were dried for approximately two (2) minutes. Steps were repeated for all pans to coat all tablets in the batch and film coated until the tablet weight has increased by 2.0% to 4.5%. All tablets were packaged in plastic bottles with desiccants, and the bottles were heat sealed, then placed under the stress condition.

### Example 7

### Stability Studies

The stability of Form V of sitagliptin phosphate bulk material and tablets is assessed by storing samples for up to 12 weeks at 25° C./60% RH or up to 3 weeks at 40° C./75% RH. Changes are monitored using a stability-indicating HPLC method. Results were calculated by normalized peak area (npa). Degradants are identified by comparison of their relative retention times against impurity standards.

### (i) Polymorph Form V of Sitagliptin Phosphate Bulk Material

Polymorph Form V of sitagliptin phosphate bulk material was stable with respect to polymorphic form (or phase) stability as well as formation of known and unknown degradants for over 3 months when stored under normal conditions of temperature and humidity. Similarly, polymorph (phase) and chemical stability of Form V was demonstrated at elevated temperatures and humidity (40° C./75%) for over 3 weeks.

### ii) Tablets Comprising Form V of Sitagliptin Phosphate

Tablets comprising polymorph Form V of sitagliptin phosphate was stable with respect to the formation of known and unknown degradants for over 3 to 6 months when stored under normal manufacturing and storage conditions of temperature and humidity (25° C./65% relative humidity).

We claim:

1. Polymorph Form V of crystalline sitagliptin phosphate.

2. The polymorph Form V of claim 1, characterized as having an X-ray diffraction pattern with characteristic peaks (expressed in 2θ±0.2° 2θ) at one or more of the following positions: 4.60, 9.32, 12.38, 13.40, 13.92, 18.24, 23.60, 24.36, 25.40 or 26.60.

3. The polymorph Form V of claim 1, characterized as having X-ray powder diffraction pattern substantially the same as that shown in FIG. 1, FIG. 2 or FIG. 3.

4. The polymorph Form V of claim 1, characterized as having an endothermic peak at about 210-214° C. in differential scanning calorimetry (DSC) and being substantially the same as that shown in FIG. 4 or FIG. 5.

5. A composition comprising (a) polymorph Form V of crystalline sitagliptin phosphate and (b) a crystalline, hydrate, solvate, amorphous, polymorph Form I, Form II, Form III, Form IV or other polymorphic forms of sitagliptin phosphate other than Form V, wherein the total weight of sitagliptin phosphate in the composition is the sum of (a) and (b).

6. The composition of claim 5, wherein the composition comprising less than 0.1% to at least 99.9% by weight of polymorph Form V based on the total weight of sitagliptin phosphate in the composition.

7. The composition of claim 5, wherein the composition comprising less than 0.1% by weight of polymorph Form V based on the total weight of sitagliptin phosphate in the composition.

8. The composition of claim 5, wherein the composition comprising less than 2% by weight of polymorph Form V based on the total weight of sitagliptin phosphate in the composition.

9. The composition of claim 5, wherein the composition comprising at least 50% by weight of polymorph Form V based on the total weight of sitagliptin phosphate in the composition.

10. The composition of claim 5, wherein the composition comprising at least 80% by weight of polymorph Form V based on the total weight of sitagliptin phosphate in the composition.

11. The composition of claim 5, wherein the composition comprising at least 95% by weight of polymorph Form V based on the total weight of sitagliptin phosphate in the composition.

12. The composition of claim 5, wherein the composition comprising at least 99.9% by weight of polymorph Form V based on the total weight of sitagliptin phosphate in the composition.

13. A pharmaceutical composition comprising polymorph Form V of crystalline sitagliptin phosphate with one or more pharmaceutically acceptable carriers, excipients, diluents, additives, fillers, lubricants or binders.

14. The pharmaceutical composition of claim 13, wherein sitagliptin phosphate comprising less than 0.1% to at least 99.9% by weight of polymorph Form V based on the total weight of sitagliptin phosphate in the pharmaceutical composition.

15. The pharmaceutical composition of claim 13, wherein the composition is formulated for oral administration.

16. The pharmaceutical composition of claim 15, wherein the dosage form is a tablet or a capsule.

17. The pharmaceutical composition of claim 16, wherein the dosage form is a delayed, sustained or extended release formulation.

18. The pharmaceutical composition of claim 17, wherein the rate controlling excipient is selected from a group consisting of hydroxyalkyl celluloses, poly(ethylene) oxides, alkyl cellulose, carboxymethyl celluloses, carboxyvinyl polymers, hydrophilic cellulose derivatives, polyethylene glycol derivatives or polyvinylpyrrolidone derivatives.
